(19) United States
(12) Patent Application Publication
(10) Pub. No.: US 2018/0344602 A1
(43) Pub. Date: Dec. 6, 2018

(54) AQUEOUS COMPOSITION CONTAINING OMEGA-AMINO CARBOXYLIC ACID ESTERS AND FATTY ALCOHOL

(71) Applicant: Evonik Degussa GmbH, Essen (DE)

(72) Inventors: Dominik Schuch, Düsseldorf (DE); Christian Hartung, Essen (DE); Hans Henning Wenk, Mülheim an der Ruhr (DE); Oliver Springer, Wesel (DE); Peter Schwab, Essen (DE); Peter Muss, Essen (DE)

(21) Appl. No.: 15/562,309

(22) PCT Filed: Apr. 25, 2016
(86) PCT No.: PCT/EP2016/059142

§ 371 Date: Sep. 27, 2017

(30) Foreign Application Priority Data
May 21, 2015 (EP) 15168565.8

(51) Int. Cl. A61K 8/44; A61Q 5/12; A61Q 5/06; A61Q 5/10; A61K 8/34; A61K 8/06

(52) U.S. Cl. CPC A61K 8/44; A61Q 5/12; A61K 8/06; A61Q 5/10; A61K 8/342; A61Q 5/06

(57) ABSTRACT

The invention relates to aqueous compositions comprising ω-aminocarboxylic acid ester and fatty alcohol. The ω-aminocarboxylic acid esters may have the general formula 1, the general formula 2 and the general formula 3, as follows:

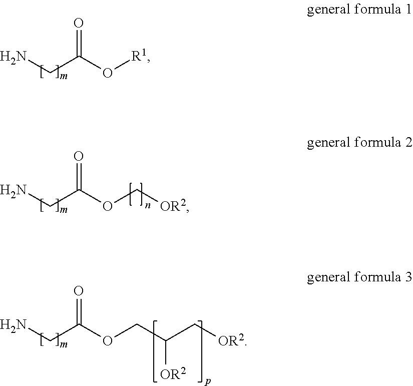

### FIELD OF THE INVENTION

The invention relates to aqueous compositions comprising ω-aminocarboxylic acid ester and fatty alcohol.

### PRIOR ART

The use of compounds that contain amino groups as conditioners for skin and hair in cosmetics has been widespread for a long time.

WO2011002746, for example, describes α-amino acid esters as conditioners.

It was an object of the invention to provide new compositions with exceptional conditioning properties.

### DESCRIPTION OF THE INVENTION

Surprisingly, it has been found that the compositions described below have excellent application properties.

The present invention therefore provides aqueous compositions comprising, as component A), ω-aminocarboxylic acid ester and, as component B), fatty alcohol.

The present invention further provides the use of ω-aminocarboxylic acid esters for conditioning skin and keratin fibers, in particular hair.

The present invention further provides the use of ω-aminocarboxylic acid esters as emulsifier, in particular a cationic emulsifier, in preferably cosmetic formulations.

An advantage of the present invention is that the composition according to the invention can be provided completely on the basis of natural raw materials.

A further advantage of the present invention is that the conditioning effect can be brought about without using permanent quat structures.

A further advantage is that the ω-aminocarboxylic acid esters used have improved hydrolysis stability.

A further advantage of the present invention is that a good conditioning performance can be achieved without a negative influence on the formulation viscosity.

A further advantage is that the ω-aminocarboxylic acid esters used can also be used in shampoos and thereby have a conditioning action.

A further advantage is that the ω-aminocarboxylic acid esters used have good biodegradability. A further advantage of the present invention is that the shine of the treated keratin fibers is increased.

A further advantage of the present invention is that the ω-aminocarboxylic acid esters used develop a good effect even when used in small amounts.

A further advantage is that the ω-aminocarboxylic acid esters used have little impact from an ecological point of view.

A further advantage is that the ω-aminocarboxylic acid esters used exhibit an easier ability to be washed out on keratin fibers than quaternary ester compounds known hitherto.

A further advantage of the present invention is that the ω-aminocarboxylic acid esters used do not crystallize out.

A further advantage of the present invention is that it protects hair colorant from washing out.

A further advantage of the present invention is that the ω-aminocarboxylic acid esters can be formulated more easily.

A further advantage of the present invention is that the ω-aminocarboxylic acid esters reduce combing forces on wet and dry hair.

A further advantage is that the ω-aminocarboxylic acid esters used can be used in high active substance contents.

A further advantage of the present invention is that the ω-aminocarboxylic acid esters used produce, as emulsifiers in O/W emulsions, an exceptional skin feel.

A further advantage of the present invention is that it is particularly economical.

In connection with the present invention, the term “aqueous” is to be understood as meaning a composition which comprises at least 5% by weight, preferably at least 30% by weight, in particular at least 70% by weight, of water, based on the total composition under consideration. In connection with the present invention, the “pH” is defined as the value which is measured for the corresponding substance at 25° C. after stirring for five minutes using a pH electrode calibrated in accordance with ISO 4319 (1977).

The term “ω-aminocarboxylic acid esters” is to be understood as meaning both the esters and also their salts. The salts are present under aqueous conditions in particular protonated at the w-amino group.

ω-Aminocarboxylic acid esters which may be present in the compositions according to the invention are, for example, esterification products of ω-aminocarboxylic acids with at least one alcohol, such as, for example, fatty alcohols and polyols, such as, for example, alkylene glycols, glycerol, polyglycerol, or mixtures thereof. If polyols have been esterified with the ω-aminocarboxylic acid, then further carboxylic acids, e.g. fatty acids, can be esterified in with the ω-aminocarboxylic acid ester which is present in the compositions according to the invention.

Preferably present ω-aminocarboxylic acid esters are selected from the group comprising, preferably consisting of, ω-aminocarboxylic acid esters of the general formula 1, the general formula 2 and the general formula 3,

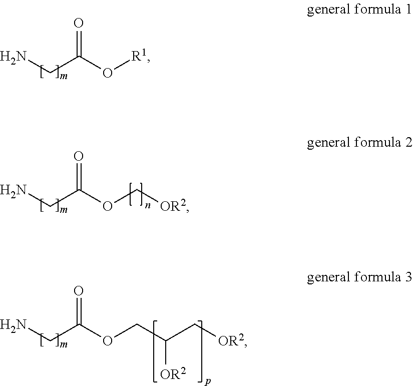

where
m=5 to 21, preferably 7 to 17, particularly preferably 11,
n=2 to 12, preferably 3 to 6,
p=1 to 10, preferably 1 to 4, particularly preferably 1,
R¹=linear or branched alkyl radical having 6 to 22, preferably 8 to 18, particularly preferably 12 to 18, carbon atoms and optionally having unsaturated bonds,
R²=independently of one another, H or a linear or branched acyl radical having 6 to 22, preferably 8 to 18, particularly preferably 12 to 18, carbon atoms optionally having unsaturated bonds, or an ω-aminoacyl radical, in particular

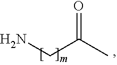

where m is as defined above.

Particularly preferably present ω-aminocarboxylic acid esters are selected from ω-aminocarboxylic acid esters of the general formula 1

where
m=11 and
R¹=selected from lauryl, myristyl, palmityl, oleyl and stearyl, and ω-aminocarboxylic acid esters of the general formula 3
where
m=11, p=1,
R²=selected from H, lauroyl, myristoyl, palmitoyl, oleoyl, stearoyl and

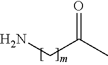

where m=11.

Component A) is present in the composition according to the invention preferably in an amount of from 0.1 to 7% by weight, preferably 0.2 to 5% by weight and particularly preferably 0.3 to 4% by weight, where the percentages by weight refer to the total composition.

Under aqueous conditions, at least some of the ω-aminocarboxylic acid ester is present protonated at the ω-amino group, depending on the pH. Anions are present as counterion of this ω-aminocarboxylic acid ester in the composition according to the invention, in particular chloride, bromide, iodide, alkyl sulphate, e.g. methyl sulphate, ethyl sulphate, alkylsulphonate, e.g. mesylate or esylate, triflate, tosylate, phosphate, sulphate, hydrogen sulphate, lactate, glycolate, acetate and citrate.

The compositions according to the invention comprise at least one fatty alcohol (component B). Fatty alcohol in this context is preferably understood as meaning an unbranched or branched monoalcohol with an alkyl group of 8 to 30 carbon atoms, which may also be unsaturated. Preferred fatty alcohols are octanol, decanol, lauryl alcohol, isolauryl alcohol, anteisolauryl alcohol, myristyl alcohol, isomyristyl alcohol, cetyl alcohol, palmoleyl alcohol, stearyl alcohol, isostearyl alcohol, anteisostearyl alcohol, eicosanol, petroselinyl alcohol, Guerbet alcohol, arachyl alcohol, gadoleyl alcohol, behenyl alcohol, erucyl alcohol, hectacosanol, octacosanol, and melissyl alcohol, and mixtures thereof, in particular technical-grade mixtures, preferably technical-grade coconut or tallow fatty alcohols having 12 to 18, preferably having 16 to 18, carbon atoms, and also the monounsaturated fatty alcohols, such as oleyl alcohol, elaidyl alcohol, delta-9-cis-hexadecenol, delta-9-octadecenol, trans-delta-9-octadecenol, cis-delta-11-octadecenol, trans-10,cis-12-hexadecadien-1-ol, octacosa-10,19-dien-1-ol and polyunsaturated fatty alcohols such as e.g. linoleyl alcohol (9Z,12Z-octadecadien-1-ol), elaidolinoleyl alcohol (9E,12E-octadecadien-1-ol), linolenyl alcohol (9Z,12Z,15Z-octadecatrien-1-ol), elaidolinolenyl alcohol (9E,12E,15E-octadecatrien-1-ol), where mixtures of coconut or tallow fatty alcohols having 16 to 18 carbon atoms are particularly preferred.

Preference is given to a fatty alcohol which has a melting point greater than 25° C., particularly preferably greater than 50° C., at a pressure of 1 bar.

The fatty alcohol is preferably present in an amount of 0.5 to 20% by weight, preferably 1 to 10% by weight, in particular 2 to 7% by weight, in the composition according to the invention, where the percentages by weight refer to the total composition.

It has proven to be advantageous if the compositions according to the invention additionally comprise a component C) emulsifier, in particular in an amount of 0.1 to 10% by weight, preferably 0.25 to 5% by weight, in particular 0.5 to 2.0% by weight, where the percentages by weight refer to the total composition.

Preferably present emulsifiers are nonionic emulsifiers.

Emulsifiers preferred in this context are selected from the group of the fatty alcohol alkoxylates, in particular the fatty alcohol ethoxylates. Particularly preferred fatty alcohol ethoxylates present are selected from the group comprising polyoxyethylene ethers of lauryl alcohol, CAS number 9002-92-0, macrogol lauryl ether, e.g. polyoxyethylene (4) lauryl ether (Laureth-4, INCI),

polyoxyethylene (9) lauryl ether Laureth-9 (INCI),
polyoxyethylene (23) lauryl ether Laureth-23 (INCI)
polyoxyethylene ethers of cetyl alcohol, CAS number 9004-95-9, e.g.
polyoxyethylene (2) cetyl ether Ceteth-2 (INCI),
polyoxyethylene (10) cetyl ether Ceteth-10 (INCI)
polyoxyethylene (20) cetyl ether Ceteth-20 (INCI)
polyoxyethylene ethers of cetylstearyl alcohol, CAS number 68439-49-6, e.g.
polyoxyethylene (6) cetylstearyl ether Ceteareth-6 (INCI)
polyoxyethylene (20) cetylstearyl ether Ceteareth-20 (INCI)
polyoxyethylene (25) cetylstearyl ether Ceteareth-25 (INCI)
polyoxyethylene ethers of stearyl alcohol, CAS number 9005-00-9, e.g.
polyoxyethylene (2) stearyl ether Steareth-2 (INCI)
polyoxyethylene (10) stearyl ether Steareth-10 (INCI)
polyoxyethylene (20) stearyl ether Steareth-20 (INCI)
polyoxyethylene ethers of oleyl alcohol, CAS number 9004-98-2, e.g.
polyoxyethylene (2) oleyl ether Oleth-2 (INCI)
polyoxyethylene (10) oleyl ether Oleth-10 (INCI)
polyoxyethylene (20) oleyl ether Oleth-20 (INCI)
or
polyoxyethylene (10) tridecyl ether (CAS number 24938-91-8) and Trideceth-10 (INCI).

Alternatively preferred emulsifiers are selected from the group of polyol esters, in particular the glycerol esters and polyglycerol esters, in particular the polyglycerol esters. Preferably present (poly)glycerol esters are characterized in that they are partial esters. Particularly preferred polyglycerol partial esters are selected from the group comprising polyglycerol partial esters as described in EP-B-0 835 862, which are obtainable by esterification of a polyglycerol mixture with a degree of esterification of the polyglycerol between 30 and 75% and saturated or unsaturated, linear or branched fatty acids with 12 to 22 carbon atoms and dimer fatty acids with an average functionality of 2 to 2.4, esters of citric acid such as, for example, the 0/W emulsifier glyceryl stearate citrate, (2-hydroxy-1,2,3-propanetricarboxylic acid-1,2,3-propanetriol monooctadecanoate, INCI Glyceryl Stearate Citrate, CAS 39175-72-9), the citric acid ester of glyceryl stearate, commercially available inter alia under the name AXOL C 62, glyceryl stearate citrate as described in WO2006034992 and WO2008092676 and glyceryl oleate citrate as described in WO2004112731, likewise simple polyglycerol esters, such as, for example, polyglycerol-3 distearate, polyglyceryl-10 stearate, polyglyceryl-6 distearate, mixed esters of polyglycerol and methyl glucose and stearic acid, such as, for example, polyglyceryl-3 methyl glucose distearate and (poly)glycerol partial esters with one or more carboxylic acids having 10 to 24 carbon atoms and residues of a polyfunctional carboxylic acid.

In principle, sorbitan or sucrose esters can also be used as polyol esters. A customary combination is, for example, Sorbitan Stearate & Sucrose Cocoate.

Emulsifiers preferably present in a further alternative are selected from the group of modified siloxanes, for example those which also carry polyethers besides aliphatic groups based on alpha-olefins. Siloxane-based emulsifiers for oil-in-water emulsions must have a hydrophilic character, for which reason they are generally pure polyether siloxanes. Particularly suitable examples are relatively hydrophobic polyethersiloxanes as described in EP 1125574, high molecular weight polyethersiloxanes as described in EP2168564 and organomodified siloxane block copolymers as described in WO2009138306. Preferably present modified siloxanes are characterized in that they have an HLB value>8. Particularly preferred modified siloxanes are selected from the group comprising Bis-PEG/PPG-16/16 Dimethicone, PEG/PPG-16/16 Dimethicone, Bis-PEG/PPG-20/5 PEG/PPG-20/5 Dimethicone and Methoxy PEG/PPG-25/4 Dimethicone.

In connection with the present invention, the aforementioned emulsifiers produce particularly storage-stable formulations.

Preferred compositions according to the invention are emulsions, preferably oil-in-water emulsions, particularly preferably emulsions in which the oil phase is solid at 25° C., an oil-in-water suspension thus being present at 25° C.

The compositions according to the invention advantageously have, at 25° C., a pH of from 1 to 6.9, particularly preferably from 2 to 6.5, in particular from 2.5 to 6.

It has proven to be advantageous if the compositions according to the invention additionally comprise a component D) surfactant, in particular in an amount of from 0.1 to 10% by weight, preferably 0.25 to 5% by weight, in particular 0.5 to 2.0% by weight, where the percentages by weight refer to the total composition.

In the context of the present invention, the term “surfactant” is understood as meaning organic substances with interface-active properties which have the ability to reduce the surface tension of water at 20° C. and at a concentration of 0.5% by weight, based on the total composition, to below 45 mN/m. The surface tension is determined here by the ring method in accordance with DuNoüy at 25° C.

The surfactants are in particular nonionic surfactants, anionic surfactants, cationic surfactants and amphoteric (zwitterionic) surfactants.

Preference is given to compositions according to the invention which are characterized in that the surfactant is selected from the group comprising, preferably consisting of:

anionic surfactants, cationic surfactants and amphoteric surfactants, with anionic surfactants and cationic surfactants being particularly preferred.

If the composition according to the invention comprises an anionic surfactant, preference is given in particular to compositions according to the invention which are characterized in that the anionic surfactant is selected from the group comprising, preferably consisting of:

alkyl sulphates in the form of their alkali metal, ammonium or alkanolammonium salts, alkyl ether sulphates, alkyl phosphates in the form of their alkali metal, ammonium or alkanolammonium salts, alkyl ether carboxylates in the form of their alkali metal or ammonium salts, acyl sarcosinates in the form of their alkali metal or ammonium salts, sulfosuccinates in the form of their alkali metal or ammonium salts and acylglutamates in the form of their alkali metal or ammonium salts, with alkyl sulphates and alkyl ether sulphates being particularly preferred.

If the composition according to the invention comprises a cationic surfactant, preference is given in particular to compositions according to the invention which are characterized in that the cationic surfactant is selected from the group of the quaternary ammonium compounds, preferably consisting of alkyltrimethylammonium compounds, with palmitamidopropyltrimonium chloride being particularly preferred.

If the composition according to the invention comprises an amphoteric surfactant, preference is given in particular to compositions according to the invention which are characterized in that the amphoteric surfactant is selected from the group comprising, preferably consisting of: betaines, amphoacetates and amphopropionates, N-alkyl-N,N-dimethylammoniumglycinates, for example cocoalkyldimethylammonium glycinate, N-acylaminopropyl-N,N-dimethylammonium glycinates, for example cocoacylaminopropyldimethylammonium glycinate, 2-alkyl-3-carboxylmethyl-3-hydroxyethylimidazolines having in each case 8 to 18 carbon atoms in the alkyl or acyl group, and also cocoacylaminoethylhydroxyethyl carboxymethylglycinate, compounds which, apart from a C8/18-alkyl or -acyl group in the molecule, comprise at least one free amino group and at least one —COOH— or —SO₃H group and are capable of forming internal salts, such as, for example, N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case about 8 to 18 carbon atoms in the alkyl group, N-cocoalkylaminopropionate, cocoacylaminoethylaminopropionate and C12/18-acylsarcosine, with N-acylaminopropyl-N,N-dimethylammonium glycinates being particularly preferred.

The compositions according to the invention can comprise e.g. at least one further, additional component selected from the group of
emollients,
coemulsifiers,
thickeners/viscosity regulators/stabilizers,
antioxidants,
hydrotropes (or polyols),
solids and fillers,
pearlescence additives,
deodorant and antiperspirant active ingredients,
insect repellents,
self-tanning agents,
preservatives,
conditioners,
perfumes,
dyes,
cosmetic active ingredients,
care additives,
superfatting agents,
solvents.

Substances which can be used as exemplary representatives of the individual groups are known to the person skilled in the art and can be found for example in the German application DE 102008001788.4. This patent application is herewith incorporated as reference and thus forms part of the disclosure.

As regards further optional components and the amounts used of these components, reference is made expressly to the relevant handbooks known to the person skilled in the art, e.g. K. Schrader, “Grundlagen and Rezepturen der Kosmetika [Fundamentals and formulations of cosmetics]”, 2nd edition, pages 329 to 341, Hüthig Buch Verlag Heidelberg.

The amounts of the particular additives are governed by the intended use.

Typical guide formulations for the respective applications are known prior art and are contained for example in the brochures of the manufacturers of the particular basic materials and active ingredients. These existing formulations can usually be adopted unchanged. If necessary, the desired modifications can, however, be undertaken without complication by means of simple experiments for the purposes of adaptation and optimization.

The ω-aminocarboxylic acid esters and the ω-aminocarboxylic acid esters which are preferably present in the compositions according to the invention, and the compositions according to the invention can be used according to the invention for the cosmetic treatment of skin or keratin fibers, especially for treating hair.

In this connection, preference is given to using those compounds of the general formulae 1, 2 and 3 which are described above as being preferably present in the compositions according to the invention.

The use according to the invention leads to the improvement in the conditioning, shine, flexibility, elasticity and/or combability, and also to a reduction in the probability of breakage of the treated fibers and, moreover, it reduces the antistatic forces between the fibers.

The present invention further provides the use of ω-aminocarboxylic acid esters, and also the co-aminocarboxylic acid esters which are preferably present in the compositions according to the invention as emulsifier, in particular cationic emulsifier, in preferably cosmetic formulations, in particular to give O/W emulsions.

The examples listed below describe the present invention by way of example without any intention of limiting the invention, the scope of application of which arises from the entire description and the claims, to the embodiments specified in the examples.

### EXAMPLES

### 1: Preparation of ω-aminocarboxylic Acid Esters According to the Invention

1.1. Preparation of 12-aminolauric Acid Lauryl Ester MU4312:

Under a nitrogen atmosphere, a mixture of 52.6 g of 12-aminolauric acid (1 mol equivalent) and 93.9 g of lauryl alcohol (2 mol equivalents) was admixed at 90° C. with stirring with a solution of 24.9 g of methanesulphonic acid (1.05 mol equivalents) in 10.3 g of water. Then, the reaction mixture was stirred for 10 hours at 140° C. and water was continuously distilled off. After cooling to room temperature, the reaction product was in the form of a yellowish to brownish colored solid.

1.2. Preparation of 12-aminolauric Acid Palmityl Ester MU4343:

Under a nitrogen atmosphere, a mixture of 48.3 g of 12-aminolauric acid (1 mol equivalent) and 108.8 g of palmityl alcohol (2 mol equivalents) was admixed at 90° C. with stirring with a solution of 22.9 g of methanesulphonic acid (1.05 mol equivalents) in 9.5 g of water. Then, the reaction mixture was stirred for 10 hours at 140° C. and water was continuously distilled off. After cooling to room temperature, the reaction product was in the form of a yellowish to brownish colored solid.

1.3. Preparation of 12-aminolauric Acid Stearyl Ester MU4342:

Under a nitrogen atmosphere, a mixture of 45.2 g of 12-aminolauric acid (1 mol equivalent) and 113.5 g of stearyl alcohol (2 mol equivalents) was admixed at 90° C. with stirring with a solution of 21.4 g of methanesulphonic acid (1.05 mol equivalents) in 8.8 g of water. Then, the reaction mixture was stirred for 10 hours at 140° C. and water was continuously distilled off. After cooling to room temperature, the reaction product was in the form of a yellowish to brownish colored solid.

1.4. Preparation of 12-aminolauric Acid Oleyl Ester MU4313:

Under a nitrogen atmosphere, a mixture of 45.5 g of 12-aminolauric acid (1 mol equivalent) and 112.9 g of oleyl alcohol (2 mol equivalents) was admixed at 90° C. with stirring with a solution of 21.5 g of methanesulphonic acid (1.05 mol equivalents) in 8.9 g of water. Then, the reaction mixture was stirred for 10 hours at 140° C. and water was continuously distilled off. After cooling to room temperature, the reaction product was in the form of a yellowish to brownish colored solid.

1.5. Preparation of 12-aminolauric Acid Glyceryl Laurate Ester AZMU370:

Under a nitrogen atmosphere, a mixture of 65.5 g of 12-aminolauric acid (1 mol equivalent) and 83.5 g of glyceryl laurate (1.1 mol equivalent) was admixed at 90° C. with stirring with a solution of 31.0 g of methanesulphonic acid (1.05 mol equivalents) in 12.8 g of water. Then, the reaction mixture was stirred for 10 hours at 140° C. and water was continuously distilled off. After cooling to room temperature, the reaction product was in the form of a yellowish to brownish colored solid.

1.6. Preparation of 12-aminolauric Ester MU4346 with 1,3-Propanediol:

Under a nitrogen atmosphere, a mixture of 98.1 g of 12-aminolauric acid (1 mol equivalent) and 35.4 g of 1,3-propanediol (2 mol equivalents) was admixed at 90° C. with stirring with a solution of 46.5 g of methanesulphonic acid (1.05 mol equivalents) in 19.9 g of water. Then, the reaction mixture was stirred for 10 hours at 140° C. and water was continuously distilled off. After cooling to room temperature, the reaction product was in the form of a yellowish to brownish colored solid.

1.7. Preparation of 12-aminolauric Acid Glyceryl Ester MU4347:

Under a nitrogen atmosphere, a mixture of 94.7 g of 12-aminolauric acid (1 mol equivalent) and 40.5 g of glycerol (2 mol equivalents) was admixed at 90° C. with stirring with a solution of 44.8 g of methanesulphonic acid (1.05 mol equivalents) in 30.4 g of water. Then, the reaction mixture was stirred for 10 hours at 140° C. and water was continuously distilled off. After cooling to room temperature, the reaction product was in the form of a yellowish to brownish colored solid.

### 2: Comparative Examples not According to the Invention

### 2.1. Behentrimonium Chloride

VARISOFT® BT 85 (INCI: Behentrimonium Chloride. Evonik Industries AG), 85% strength with 15% isopropanol.

2.2. Brassicyl Isoleucinate Esylate (and) Brassica Alcohol:

Emulsense HC (INCI: Brassicyl Isoleucinate Esylate (and) Brassica Alcohol. Inolex), approx. 25% strength cationic in approx. 75% Brassica Alcohol.

### 3: Application Examples: Testing of the Conditioning of Hair by Means of Sensory Tests in a Hair Rinse

For the performance assessment of the conditioning of hair, the compounds according to the invention from synthesis examples 1.1, 1.4 and 1.5, as well as the commercially available products 2.1 and 2.2, were used in a simple cosmetic hair rinse formulation and compared.

The performance properties on use in hair rinses were verified in the following formulas:

| | Formulation examples | | | | | |
|---|---|---|---|---|---|---|
| | 0a | 1a | 2a | 3a | C4a | C5a |
| TEGINACID ® C, Evonik | 0.5% | 0.5% | 0.5% | 0.5% | 0.5% | 0.5% |
| Industries (INCI: Ceteareth-25) | | | | | | |
| TEGO ® Alkanol 16, Evonik | 5.0% | 5.0% | 5.0% | 5.0% | 5.0% | 5.0% |
| Industries (INCI: Cetyl Alcohol) | | | | | | |
| Neolone PE, The Dow Chemical | 0.45% | 0.45% | 0.45% | 0.45% | 0.45% | 0.45% |
| Company (INCI: | | | | | | |
| Phenoxyethanol; | | | | | | |
| Methylisothiazolinone) | | | | | | |
| Water, demineralized | ad 100.0% | | | | | |
| Sodium hydroxide solution | ad pH 4.3 ± 0.3 | | | | | |
| (10% in water) | | | | | | |
| Synthesis example 1.1, | | 1.0% | | | | |
| 12-Aminolauric acid lauryl ester | | | | | | |
| (according to the invention) | | | | | | |
| Synthesis example 1.4, | | | 1.0% | | | |
| 12-Aminolauric acid oleyl ester | | | | | | |
| (according to the invention) | | | | | | |
| Synthesis example 1.5, | | | | 1.0% | | |
| 12-Aminolauric acid glyceryl | | | | | | |
| laurate ester (according to the | | | | | | |
| invention) | | | | | | |
| VARISOFT ® BT 85 (INCI: | | | | | 1.2% | |
| Behentrimonium Chloride) 85% | | | | | | |
| strength in isopropanol | | | | | | |
| (not according to the invention) | | | | | | |
| Emulsense HC (INCI: Brassicyl | | | | | | 4.0% |
| Isoleucinate Esylate (and) | | | | | | |
| Brassica Alcohol), 25% strength | | | | | | |
| in Brassica Alcohol. | | | | | | |
| (not according to the invention) | | | | | | |

The hair rinses were produced here as is generally customary by heating the oil phase of fatty alcohol (Cetyl Alcohol), emulsifier (Ceteareth-25) and the organic conditioners according to the invention or not according to the invention until melting (˜75° C.), and then adding this oil phase to the water phase (water) at 75° C. with stirring. The mixture was homogenized for 30 seconds and then slowly cooled with gentle stirring.

The pH was then adjusted with sodium hydroxide solution.

However, for comparison product 2.2, in a different and very involved method, only the water phase had to be adjusted to pH˜12 before homogenization (either with NaOH solution or, as described in WO2011002746, with L-arginine), since otherwise an inhomogeneous emulsion was formed later during the pH adjustment. That is to say, depending on the composition of the formulation, in most cases the pH thus has to be adjusted twice for comparison product 2.2, or it has to be tested at length what pH has to be established for the water phase in order to ultimately arrive at the intended pH. Despite the high cationic active ingredient content of 100%, the products according to the invention can thus be processed considerably better than the amino acid ester example 2.2 of the prior art since this laborious pH adjustment is not necessary for the products according to the invention and no inhomogeneous emulsions are formed.

For the performance assessment, hair tresses which are used for sensory tests were subjected to standardized predamaging by means of a bleaching treatment. For this, customary hairstyling products were used. The test procedure, the base materials used and the details of the assessment criteria are described in DE 103 27 871.

The hair was pretreated using a shampoo which contained no conditioners.

Standardized treatment of predamaged hair tresses with conditioning formulations:

The hair tresses predamaged as described above were treated as follows with the above-described conditioning rinse:

The hair tresses were wetted under running warm water. The excess water was gently squeezed out by hand, then the non-conditioning shampoo is applied and gently worked into the hair (1 ml/hair tress (2 g)). After a contact time of 1 min, the hair was rinsed for 1 min. Directly afterwards, the rinse to be tested was applied and gently worked into the hair (1 ml/hair tress (2 g)). After a contact time of 1 min, the hair was rinsed for 1 min with water.

### Assessment Criteria:

The sensory evaluations of the hair tresses were carried out by means of grades awarded on a scale from 1 to 5, with 1 being the worst evaluation and 5 being the best evaluation. The individual test criteria were each given their own evaluation.

The test criteria are as follows: Wet combability, wet feel, rinsability, dry combability, dry feel, appearance/shine.

The table below compares the results of the sensory assessment of the treatment of the hair tresses carried out as described above with the formulations 1a, 2a and 3a according to the invention, the comparison formulations C4a and C5a, and the control formulation 0a (placebo without test substance).

| | Wet | Wet | | Dry | | |
|---|---|---|---|---|---|---|
| | combability | feel | Rinsability | combability | Dry feel | Shine |
| Control formulation 0a | 1.9 | 1.9 | 4.0 | 3.5 | 3.6 | 2.0 |
| Formulation 1a according | 4.1 | 4.0 | 3.5 | 4.1 | 4.1 | 3.5 |
| to the invention | | | | | | |
| Formulation 2a according | 4.0 | 4.0 | 3.3 | 4.3 | 4.3 | 3.6 |
| to the invention | | | | | | |
| Formulation 3a according | 4.0 | 4.0 | 3.2 | 4.4 | 4.3 | 3.6 |
| to the invention | | | | | | |
| Comparison formulation | 3.8 | 3.7 | 2.8 | 4.3 | 4.2 | 3.3 |
| C4a (not according to the | | | | | | |
| invention) | | | | | | |
| Comparison formulation | 3.9 | 3.6 | 2.6 | 4.3 | 4.3 | 3.2 |
| C5a (not according to the | | | | | | |
| invention) | | | | | | |

The formulations 1a, 2a and 3a according to the invention with the compounds according to the invention from synthesis examples 1.1, 1.4 and 1.5 exhibited good cosmetic evaluations in the sensory assessment. The control formulation 0a without cationic active ingredient was significantly improved by the addition of 1.0% active substance. In this connection, the already very good properties of the comparison formulations C4a and C5a particularly as regards wet feel were yet further increased by the formulations according to the invention. The wet and dry combability, and the dry feel are at a similarly high level for all products. The significantly better assessment of the formulations 1a, 2a and 3a according to the invention for shine of the hair and particularly also for the rinsability is striking. The ease of rinsability of the conditioner formulations with the compounds according to the invention is particularly surprising compared to the comparison formulations C4a and C5a. During use, this may be a major advantage (in terms of time and ecologically) since the consumer has the feeling after just a short rinsing time with water that the product has been washed out.

### 4: Further Formulation Examples

The formulation examples given in the tables below show exemplary representatives of a large number of possible compositions according to the invention.

Unless stated otherwise, the data in the tables below are data in percent by weight.

### Formulation Example 6) Rinse-Off Conditioner

| | |
|---|---|
| Water | ad 100.00% |
| VARISOFT ® BT 85, Evonik Industries | 0.50% |
| (INCI: Behentrimonium Chloride) | |
| Synthesis example 1.1 | 1.50% |
| TEGO ® Alkanol 1618, Evonik Industries | 5.00% |
| (INCI: Cetearyl Alcohol) | |
| Sodium Hydroxide (10% in water) | ad pH 5.0 |
| Preservative, Perfume | q.s. |

### Formulation Example 7) Rinse-Off Conditioner

| | |
|---|---|
| Water | ad 100.00% |
| Synthesis example 1.1 | 1.50% |
| TEGO ® Alkanol 1618, Evonik Industries | 5.00% |
| (INCI: Cetearyl Alcohol) | |
| Lauryl alcohol, Aldrich | 0.50% |
| Glycerin | 1.00% |
| Sodium Hydroxide (10% in water) | ad pH 4.5 |
| Preservative, Perfume | q.s. |

### Formulation Example 8) Rinse-Off Conditioner

| | |
|---|---|
| Water | ad 100.00% |
| Synthesis example 1.2 | 1.50% |
| TEGO ® Alkanol 1618, Evonik Industries | 4.00% |
| (INCI: Cetearyl Alcohol) | |
| Jaguar C-162, Solvay (INCI: Hydroxypropyl Guar | 0.15% |
| Hydroxypropyltrimonium Chloride) | |
| Glycerin | 1.00% |
| Sodium Hydroxide (10% in water) | ad pH 4.8 |
| Preservative, Perfume | q.s. |

### Formulation Example 9) Rinse-Off Conditioner

| | |
|---|---|
| Water | ad 100.00% |
| Synthesis example 1.1 | 1.50% |
| Synthesis example 1.3 | 0.50% |
| TEGO ® Alkanol 1618, Evonik Industries | 5.00% |
| (INCI: Cetearyl Alcohol) | |
| Glycerin | 1.50% |
| Sodium Hydroxide (10% in water) | ad pH 4.3 |
| Preservative, Perfume | q.s. |

### Formulation Example 101 Rinse-Off Conditioner

| | |
|---|---|
| Water | ad 100.00% |
| Synthesis example 1.5 | 1.50% |
| TEGO ® Care PSC 3, Evonik Industries | 0.50% |
| (INCI: Polyglyceryl-3 Dicitrate/Stearate) | |
| TEGO ® Alkanol 1618, Evonik Industries | 5.00% |
| (INCI: Cetearyl Alcohol) | |
| Glycerin | 0.50% |
| Sodium Hydroxide (10% in water) | ad pH 4.5 |
| Preservative, Perfume | q.s. |

### Formulation Example 11) Rinse-Off Conditioner

| | |
|---|---|
| Water | ad 100.00% |
| VARISOFT ® EQ 100, Evonik Industries | 1.00% |
| (INCI: Quaternium-98 (proposed)) | |
| Synthesis example 1.4 | 2.00% |
| TEGO ® Care PSC 3, Evonik Industries | 0.80% |
| (INCI: Polyglyceryl-3 Dicitrate/Stearate) | |
| TEGO ® Alkanol 1618, Evonik Industries | 6.50% |
| (INCI: Cetearyl Alcohol) | |
| Sodium Hydroxide (10% in water) | ad pH 4.8 |
| Preservative, Perfume | q.s. |

### Formulation Example 12) Rinse-Off Conditioner

| | |
|---|---|
| Water | ad 100.00% |
| ABIL ® Quat 3272, Evonik Industries (INCI: Quaternium- | 0.50% |
| 80) | |
| Synthesis example 1.2 | 2.00% |
| TEGO ® Alkanol 1618, Evonik Industries | 4.00% |
| (INCI: Cetearyl Alcohol) | |
| Sodium Hydroxide (10% in water) | ad pH 4.5 |
| Preservative, Perfume | q.s. |

### Formulation Example 13) Rinse-Off Conditioner

| | |
|---|---|
| TEGINACID ® C, Evonik Industries (INCI: Ceteareth-25) | 0.50% |
| TEGO ® Alkanol 16, Evonik Industries (INCI: Cetyl | 2.00% |
| alcohol) | |
| ABIL ® Quat 3272, Evonik Industries (INCI: Quaternium- | 0.50% |
| 80) | |
| TEGO ® Amid S 18, Evonik Industries | 1.00% |
| (INCI: Stearamidopropyl Dimethylamine) | |
| Synthesis example 1.3 | 1.50% |
| Propylene Glycol | 1.00% |
| Citric Acid Monohydrate | ad pH 3.9 |
| Water | ad 100.00% |
| Preservative, Perfume | q.s. |

### Formulation Example 14) Rinse-Off Conditioner

| | |
|---|---|
| TEGINACID ® C, Evonik Industries (INCI: Ceteareth-25) | 0.50% |
| TEGO ® Alkanol 16, Evonik Industries (INCI: Cetyl | 5.00% |
| alcohol) | |
| TEGOSOFT ® DEC, Evonik Industries | 0.50% |
| (INCI: Diethylhexyl Carbonate) | |
| Synthesis example 1.5 | 1.00% |
| Water | ad 100.00% |
| TEGO ® Cosmo C 100, Evonik Industries (INCI: Creatine) | 0.20% |
| Propylene Glycol | 1.00% |
| Sodium Hydroxide (10% in water) | ad pH 4.8 |
| Preservative, Perfume | q.s. |

### Formulation Example 15) Rinse-Off Conditioner

| | |
|---|---|
| Water | ad 100.00% |
| VARISOFT ® 300, Evonik Industries | 2.00% |
| (INCI: Cetrimonium Chloride) | |
| VARISOFT ® BT 85, Evonik Industries | 1.00% |
| (INCI: Behentrimonium Chloride) | |
| ABIL ® OSW 5, Evonik Industries | 0.70% |
| (INCI: Cyclopentasiloxane; Dimethiconol) | |
| Synthesis example 1.1 | 1.30% |
| ABIL ® Soft AF 300, Evonik Industries | 1.00% |
| (INCI: Aminopropyl Dimethicone) | |
| TEGO ® Alkanol 1618, Evonik Industries | 5.00% |
| (INCI: Cetearyl Alcohol) | |
| Citric Acid Monohydrate | ad pH 3.8 |
| Preservative, Perfume | q.s. |

### Formulation Example 16) Rinse-Off Conditioner

| | |
|---|---|
| Water | ad 100.00% |
| ABIL ® Soft AF 100, Evonik Industries | 1.00% |
| (INCI: Methoxy PEG/PPG-7/3 Aminopropyl Dimethicone) | |
| Synthesis example 1.2 | 2.80% |
| TEGO ® Alkanol 1618, Evonik Industries | 5.00% |
| (INCI: Cetearyl Alcohol) | |
| Glycerin | 1.00% |
| Sodium Hydroxide (10% in water) | ad pH 4.6 |
| Preservative, Perfume | q.s. |

### Formulation Example 17) Rinse-Off Conditioner

| | |
|---|---|
| Water | ad 100.00% |
| VARISOFT ® BT 85, Evonik Industries | 1.00% |
| (INCI: Behentrimonium Chloride) | |
| SF 1708, Momentive | 1.50% |
| (INCI: Amodimethicone) | |
| Synthesis example 1.2 | 1.20% |
| TEGO ® Alkanol 1618, Evonik Industries | 5.00% |
| (INCI: Cetearyl Alcohol) | |
| Sodium Hydroxide (10% in water) | ad pH 4.8 |
| Preservative, Perfume | q.s. |

### Formulation Example 18) Leave-In Conditioner Spray

| | |
|---|---|
| Lactic Acid, 80% | 0.20% |
| Water | ad 100.00% |
| TEGO ® Alkanol 1618, Evonik Industries | 1.00% |
| (INCI: Cetearyl Alcohol) | |
| TEGO ® Amid S 18, Evonik Industries | 1.20% |
| (INCI: Stearamidopropyl Dimethylamine) | |
| TEGIN ® G 1100 Pellets, Evonik Industries | 0.30% |
| (INCI: Glycol Distearate) | |
| TEGO ® Care PS, Evonik Industries | 0.70% |
| (INCI: Methyl Glucose Sesquistearate) | |
| TEGOSOFT ® AC, Evonik Industries | 0.20% |
| (INCI: Isoamyl Cocoate) | |
| Synthesis example 1.1 | 2.10% |
| Sodium Hydroxide (10% in water) | ad pH 4.8 |
| Preservative, Perfume | q.s. |

### Formulation Example 19) Leave-In Conditioner Spray

| | |
|---|---|
| TAGAT ® CH 40, Evonik Industries | 2.50% |
| (INCI: PEG-40 Hydrogenated Castor Oil) | |
| TEGO ® Alkanol 1618, Evonik Industries | 0.20% |
| (INCI: Cetearyl Alcohol) | |
| Ceramide VI, Evonik Industries (INCI: Ceramide 6 II) | 0.05% |
| Perfume | 0.20% |
| Water | ad 100.00% |
| Synthesis example 1.6 | 2.50% |
| Argan Oil, DSM | 0.10% |
| LACTIL ®, Evonik Industries | 1.00% |
| (INCI: Sodium Lactate; Sodium PCA; Glycine; Fructose; | |
| Urea; Niacinamide; Inositol; Sodium benzoate; Lactic Acid) | |
| TEGO ® Retain F 50, Evonik Industries 38% | 2.30% |
| (INCI: Cocamidopropyl Betaine) | |
| Sodium Hydroxide (10% in water) | ad pH 4.8 |

### Formulation Example 20) Leave-In Conditioner Foam

| | |
|---|---|
| Synthesis example 1.1 | 1.50% |
| TAGAT ® CH 40, Evonik Industries | 0.50% |
| (INCI: PEG-40 Hydrogenated Castor Oil) | |
| Perfume | 0.30% |
| TEGO ® Retain 810, Evonik Industries | 2.00% |
| (INCI: Capryl/Capramidopropyl Betaine) | |
| Water | ad 100.00% |
| TEGO ® Cosmo C 100, Evonik Industries (INCI: Creatine) | 0.50% |
| TEGO ® Alkanol 16, Evonik Industries | 0.20% |
| (INCI: Cetyl Alcohol) | |
| TEGOCEL ® HPM 50, Evonik Industries | 0.30% |
| (INCI: Hydroxypropyl Methylcellulose) | |
| VARISOFT ® 300, Evonik Industries | 1.30% |
| (INCI: Cetrimonium Chloride) | |
| LACTIL ® Evonik Industries | 0.50% |
| (INCI: Sodium Lactate; Sodium PCA; Glycine; Fructose; | |
| Urea; Niacinamide; Inositol; Sodium benzoate; Lactic Acid) | |
| Citric acid (30% in water) | ad pH 4.0 |
| Preservative | q.s. |

### Formulation Example 21) Strong Hold Styling Gel

| | |
|---|---|
| TEGO ® Carbomer 141, Evonik Industries | 1.20% |
| (INCI: Carbomer) | |
| Water | ad 100.00% |
| NaOH, 25% | 2.70% |
| PVP/VA W-735, ISP | 16.00% |
| (INCI: PVP/VA Copolymer) | |
| Synthesis example 1.2 | 1.50% |
| TEGO ® Alkanol 16, Evonik Industries | 0.20% |
| (INCI: Cetyl Alcohol) | |
| Alcohol Denat. | 10.00% |
| TAGAT ® O 2 V, Evonik Industries | 2.00% |
| (INCI: PEG-20 Glyceryl Oleate) | |
| Perfume | 0.30% |
| ABIL ® B 88183 PH, Evonik Industries (INCI: PEG/PPG- | 0.70% |
| 20/6 Dimethicone) | |
| Preservative | q.s. |

### Formulation Example 22) Hair Colorant

| Water demineralized | ad 100.00% |
|---|---|
| TEGO ® Alkanol 1618, Evonik Industries, | 12.00% |
| (INCI: Cetearyl Alcohol) | |
| Eutanol ® G, BASF (INCI: Octyldodecanol) | 3.00% |
| REWOMID ® C 212, Evonik Industries | 1.50% |
| (INCI: Cocamide MEA) | |
| Super Hartolan ® B, Crodo (INCI: Lanolin Alcohol) | 3.00% |
| Avocado oil, Henry Lamotte (INCI: Persea Gratissima Oil) | 1.50% |
| Pristerene ® 4960, Uniquema (INCI: Stearic Acid) | 6.00% |
| EDTA BD, BASF (INCI: Disodium EDTA) | 0.10% |
| Texapon ® K12G, BASF (INCI: Sodium Lauryl Sulphate) | 0.50% |
| Propylene glycol | 5.00% |
| Timica Silver Sparkle, BASF (INCI: MICA; | 1.00% |
| Titanium Dioxide) | |
| Ammonia solution, 25% strength | 6.00% |
| 2,5-Diaminotoluene sulphate, (INCI: Toluene-2,5-Diamine) | 1.40% |
| Rodol ® RS, Jos. H. Lowenstein & Sons (INCI: | 0.30% |
| Resorcinol) | |
| HC Blue A42, (INCI: 2,4-Diaminophenoxyethanol di HCl) | 0.10% |
| Sodium sulphite | 0.50% |
| Perfume | 0.20% |
| Synthesis example 1.1 | 0.50% |

### Formulation Example 23) Hair Colorant

| Water demineralized | ad 100.00% |
|---|---|
| TEGO ® Alkanol 1618, Evonik Industries | 12.00% |
| (INCI: Cetearyl Alcohol) | |
| Super Hartolan ® B, Croda (INCI: Lanolin Alcohol) | 2.50% |
| Meadowfoam ® Seed Oil, Fanning (INCI: | 1.00% |
| Limnanthes Alba) | |
| Pristerene ® 4960, Uniquema (INCI: Stearic Acid) | 5.50% |
| EDTA BD, BASF (INCI: Disodium EDTA) | 0.10% |
| Glycerin | 5.00% |
| Texapon ® N 70, BASF (INCI: Sodium Laureth Sulphate) | 2.00% |
| Monoethanolamine | 4.00% |
| 2,5-Diaminotoluene sulphate, (INCI: Toluene-2,5-Diamine) | 0.90% |
| Rodol ® RS, Jos. H. Lowenstein & Sons (INCI: | 0.20% |
| Resorcinol) | |
| Jarocol ® 4A3MP, Vivimed Labs (INCI: | 0.60% |
| 4-Amino-M-Cresol) | |
| Rodol ® PAOC, Jos. H. Lowenstein & Sons | 0.50% |
| (INCI: 4-Amino-2-Hydroxytoluene) | |
| Uantox ® EBATE, Universal Preserv-A-Chem | 0.50% |
| (INCI: Erythorbic Acid) | |
| Perfume | 0.20% |
| Synthesis example 1.4 | 1.00% |

### Formulation Example 24) Hair Colorant

| Water demineralized | ad 100.00% |
|---|---|
| TEGO ® Alkanol 1618, Evonik Industries | 10.00% |
| (INCI: Cetearyl Alcohol) | |
| Eutanol ® G, BASF Cognis (INCI: Octyldodecanol) | 1.00% |
| REWOMID ® C 212, Evonik Industries | 2.00% |
| (INCI: Cocamide MEA) | |
| TEGIN ® VS, Evonik Industries | 5.00% |
| (INCI: Glyceryl Stearate SE) | |
| Fitoderm ®, Hispano Quimica S. A. | 1.00% |
| (INCI: Squalane) | |
| Coenzyme Q 10 | 0.10% |
| EDTA BD, BASF | 0.10% |
| (INCI: Disodium EDTA) | |
| Texapon ® K12G, BASF | 0.10% |
| (INCI: Sodium Lauryl Sulphate) | |
| Propylene glycol | 5.00% |
| Ammonia solution, 25% strength | 3.00% |
| Rodol ® ERN, Jos. H. Lowenstein & Sons | 0.30% |
| (INCI: 1-Naphthol) | |
| Imexine ® OAG, Chimex | 1.00% |
| (INCI: 2-Methyl-5-Hydroxyethylaminophenol) | |
| Colorex ® WP5, Teluca | 2.60% |
| (INCI: 1-Hydroxyethyl 4,5-Diamino Pyrazole Sulphate) | |
| Ascorbic acid | 0.30% |
| Perfume | 0.30% |
| Synthesis example 1.3 | 0.70% |

### Formulation Example 25) Hair Colorant

| Water demineralized | ad 100.00% |
|---|---|
| TEGO ® Alkanol 1618, Evonik Industries | 13.00% |
| (INCI: Cetearyl alcohol) | |
| REWOMID ® C 212, Evonik Industries | 2.00% |
| (INCI: Cocamide MEA) | |
| Super Hartolan ® B, Croda (INCI: Lanolin Alcohol) | 2.50% |
| Avocado oil, Henry Lamotte (INCI: Persea Gratissima Oil) | 2.00% |
| Pristerene ® 4960, Uniquema (INCI: Stearic Acid) | 6.00% |
| EDTA BD, BASF (INCI: Disodium EDTA) | 0.10% |
| Texapon ® K12G, BASF | 0.10% |
| (INCI: Sodium Lauryl Sulphate) | |
| Propylene glycol | 5.00% |
| Ammonia solution, 25% strength | 6.00% |
| 2,5-Diaminotoluene sulphate, (INCI: Toluene-2,5-Diamine) | 1.30% |
| Rodol ® RS, Jos. H. Lowenstein & Sons (INCI: | 0.30% |
| Resorcinol) | |
| Covastyle ® TBQ, LCW Les colorants Wackherr S.A. | 0.30% |
| (INCI: t-Butyl Hydroquinone) | |
| Perfume | 0.20% |
| Synthesis example 1.5 | 0.50% |

1. An aqueous composition comprising
A) ω-aminocarboxylic acid ester and
B) fatty alcohol.

2. The composition according to claim 1, wherein component A) is selected from ω-aminocarboxylic acid esters of the general formula 1, the general formula 2 and the general formula 3, 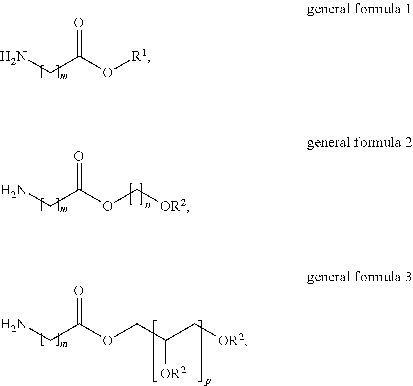
where
m is a number from 5 to 21,
n is a number from 2 to 12,
p is a number from 1 to 10,
R¹ is a linear or branched alkyl radical having 6 to 22, carbon atoms and optionally having unsaturated bonds,
R² is independently of one another, H or a linear or branched acyl radical having 6 to 22, carbon atoms and optionally having unsaturated bonds, or an ω-aminoacyl radical, in particular

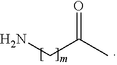

3. The composition according to claim 2, wherein component A) is selected from
ω-aminocarboxylic acid esters of the general formula 1 where
m=11 and
R¹=selected from the group consisting of lauryl, myristyl, palmityl, oleyl and stearyl, and ω-aminocarboxylic acid esters of the general formula 3
m=11, p=1,
R²=selected from the group consisting of H, lauroyl, myristoyl, palmitoyl, oleoyl, stearoyl and 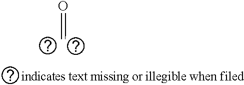 [symbol]
where m=11.

4. The composition according to claim 1, wherein A) is present in an amount of from 0.1 to 7% by weight, where the percentages by weight refer to the total composition.

5. The composition according to claim 1, additionally comprising anions selected from the group consisting of chloride, bromide, iodide, alkyl sulphate, alkylsulphonate, triflate, tosylate, phosphate, sulphate, hydrogen sulphate, lactate, glycolate, acetate and citrate.

6. The composition according to claim 1, wherein B) is present in an amount of from 0.5 to 20% by weight, in the composition, where the percentages by weight refer to the total composition.

7. The composition according to claim 1, additionally comprising
C) emulsifier, in particular nonionic emulsifier.

8. The composition according to claim 7, wherein C) is present in an amount of from 0.1 to 10% by weight, in the composition according to the invention, where the percentages by weight refer to the total composition.

9. The composition according to claim 1, having a pH of from 1 to 6.9.

10. A cosmetic treatment of skin or keratin fibers comprising the ω-aminocarboxylic acid ester as specified in claim 1.

11-15. (canceled)

16. An emulsifier comprising an ω-aminocarboxylic acid ester as specified in claim 1.

17. The composition according to claim 1 where
m is a number from 7 to 17,
n is a number from 3 to 6,
p is a number from 1 to 4,
R¹ is a linear or branched alkyl radical having 8 to 18, carbon atoms and optionally having unsaturated bonds,
R² is independently of one another, H or a linear or branched acyl radical having 8 to 18, carbon atoms and having unsaturated bonds, or an ω-aminoacyl radical, in particular

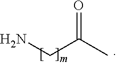

18. The composition according to claim 1 where
m is 11,
n is a number from 3 to 6,
p is 1,
R¹ is a linear or branched alkyl radical having 12 to 18, carbon atoms and optionally having unsaturated bonds,
R² is independently of one another, H or a linear or branched acyl radical having 12 to 18, carbon atoms and having unsaturated bonds, or an ω-aminoacyl radical, in particular

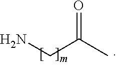

19. The composition according to claim 1, wherein A) is present in an amount of from 0.2 to 5% by weight, where the percentages by weight refer to the total composition.

20. The composition according to claim 1, wherein A) is present in an amount of from 0.3 to 4% by weight, where the percentages by weight refer to the total composition.

21. The composition according to claim 1, wherein B) is present in an amount of from 1 to 10% by weight, in the composition according to the invention, where the percentages by weight refer to the composition.

22. The composition according to claim 1, wherein B) is present in an amount of from 2 to 7% by weight, in the composition according to the invention, where the percentages by weight refer to the composition.

23. The composition according to claim 7, wherein C) is present in an amount of from 0.25 to 5% by weight, particular 0.5 to 2.0% by weight, in the composition according to the invention, where the percentages by weight refer to the total composition.

24. The composition according to claim 7, wherein C) is present in an amount of from 0.5 to 2.0% by weight, in the composition according to the invention, where the percentages by weight refer to the total composition.

25. The composition according to claim 1, having a pH of from 2 to 6.5.
